(19) United States
(12) Patent Application Publication
(10) Pub. No.: US 2013/0030355 A1
(43) Pub. Date: Jan. 31, 2013

(54) Method of use of nitrogen gas in the treatment of cancer and obesity

(71) Applicant: Donald Ray VanDeripe, Dardenne Prairie, MO (US)

(72) Inventor: Donald Ray VanDeripe, Dardenne Prairie, MO (US)

(21) Appl. No.: 13/136,207

(22) Filed: Jul. 27, 2011

(51) Int. Cl. A61M 37/00

(52) U.S. Cl. 604/26

(57) ABSTRACT

The method of the invention is directed at the injection of nitrogen gas into a tumor or fatty tissue mass previously made hypoxic/anoxic by obtunding its blood supply in order to facilitate the uptake of said nitrogen into the mitochondria of said tissues, where it becomes trapped and blocks oxygen re-uptake leading to mitochondrial apoptosis and death of local cells and tissue.

### CROSS REFERENCE TO RELATED APPLICATION

Not Applicable

### STATEMENT REGARDING FEDERALLY SPONSORED RESEARCH AND DEVELOPMENT

Not Applicable

### REFERENCE TO A MICROFICHE APPENDIX

Not Applicable

### BACKGROUND OF THE INVENTION

Cancer and obesity are two of the most widespread diseases and both are in need of improved treatment regimens. The current application selectively employs nitrogen gas as a poison to the mitochondria of the cancerous tumor tissue and to the mitochondria in the adipocytes of the fat tissue in obesity.

### BRIEF SUMMARY OF THE INVENTION

In the treatment of operable or inoperable solid tumors which are malignant or non-malignant, it is often desirable to de-bulk the tumor prior to surgical removal. As well, it is often desirable to totally eradicate the tumor without surgery. Similarly, it would be desirable to provide a treatment in obesity which locally kills the fat storing adipocytes to limit the amount of fat which can be stored in the body. The process of the current method involves making the target tissues, i.e. tumor mass or adipose tissue, ischemic so that they metabolically deplete the local oxygen reserves which leads to a drop in the tissue levels of adenosine triphosphate and a compromised integrity of the mitochondrial membrane. This allows local nitrogen gas depots to enter and fill said mitochondria and thereby block access of oxygen when blood flow is restored. Whereas the local normal physiological depots of nitrogen are adequate to fill many of the tissue mitochondria to some degree and a few completely, exogenous nitrogen must be added to completely fill all of the mitochondria of said ischemic tissue. To accomplish this, the method of this invention employs the direct injection of nitrogen gas into the ischemic mass to provide a reservoir useful in the nitrogen-mediated poisoning of all of the local tissue mitochondria.

### DETAILED DESCRIPTION OF THE INVENTION

Primary treatments for cancerous tumors include surgery, chemotherapy and radiation. Each of these can be invasive, debilitating and toxic to the patient. Chemotherapeutic agents differ among various tumors requiring different chemotherapeutic regimens for for each type of cancer, which often need to be individualized for each patient. A safe and effective alternative which could replace or reduce the need for these various therapies would represent a significant advance for the treatment of cancer. To this end the current invention can be looked upon as a safe, non-toxic alternative to chemotherapy since its goal is to kill tumor cells in a manner that allows no drug spillover into the circulation of the body excepting, perhaps, small amounts of the physiologic gas, nitrogen. The use of nitrogen in the method entails its slow infusion into an ischemic and hypoxic/anoxic tumor such that the nitrogen is actively transported into the tumor mitochondria. Once taken up into the mitochondria, nitrogen becomes trapped and it physically blocks the access of oxygen uptake resulting in a cessation of the production of adenosine triphosphate, mitochondrial membrane breakdown, apoptosis and death. The direct purpose of the nitrogen gas infusion is to evoke an intentional asphyxiation and apoptosis of the mitochondria within the tumor resulting in death of tumor cells and tissue. The treatment procedure consists of inserting into the core of the tumor a very fine small French catheter made microporous on its tip for the purpose of infusing tiny microbubbles or nanobubbles of nitrogen gas. Secondly, the tumor is made to become ischemic by means of injecting appropriate doses of vaso-spastic pharmaceuticals, e.g norepinephrine (5 micrograms) or methoxamine (0.5 milligrams), at ˜1-5 selected sites within the tumor such that essentially all tumor blood flow is obtunded for a period of 15-20 minutes. Three minutes after the injection of said vaso-spastic agents when the intra-tumor oxygen content will have been depleted, the slow infusion of the nitrogen gas bubbles will be initiated at a rate of ˜0.1-0.2m1 per minute and continued for about 10 to 15 minutes resulting in total nitrogen gas doses of from 1 ml to 3 ml. This nitrogen gas will be transported into the mitochondria where it will become trapped and block any subsequent reuptake of oxygen once the vaso-spastic agents have been metabolized and blood flow returns. This trapping of nitrogen then becomes irreversible since the return of blood flow will bring blood plasma which is fully saturated with nitrogen gas from the body water stores and inhaled from the ambient atmosphere. Therefore, without oxygen as an energy source, apoptosis and death of mitochondria will follow and cells and tissue die, and the tumor is ablated.

Alternatively, in selected cases where vascular access allows the close intra-arterial floating of a small balloon catheter, the blood flow can be stopped by balloon inflation and the nitrogen perfusion can be channeled through a microporous tip of said catheter. The same technology described above can be usefully employed to treat obesity using the percutaneous approach. The equipment and vaso-spastic agents would be the same or similar, and the doses and infusion rates of nitrogen would be in the same order of magnitude. The main difference would be that with tumors one has a single defined volume of tissue which in most cases be treated with a single procedure, but the treatment protocol for obesity would have to be repeated several or many times in order to sculpt the various fat-laded areas distributed over the body. The goal of the treatment in obesity is simply to kill the mitochondria of the adipocytes (fat cells), which are the repository of most body fat. The advantage of the method for obesity is that once killed the adipocytes are unavailable to store fat, and the return of new adipocytes may be slow or almost negligible. Then, the treatment method can be used in conjunction with exercise and diet in an overall obesity management plan to the benefit of the patient's health and well-being. In practice, one would select areas and fat tissue volumes of from 100-300 grams or more if proven safe. The upper limit of a single treatment would be based on the volumes of fat which could be safely released into the circulation.

The new technology which is the invention is clearly the administration of nitrogen gas to tumors and fat deposits of the body. The ancillary catheter technology, equipment and pharmaceuticals are known in the art and will be readily optimized based on usage needs. As well, the exact rates and doses of the nitrogen gas infusion will be determined based on animal experimentation and human use experience.

What is claimed is:

1) The slow infusion of nitrogen gas as small bubbles into a tissue mass previously made anoxic from a stoppage of blood flow by 1-5 injection(s) of a suitable vaso-spastic pharmaceutical into said tissue mass, said infusion of nitrogen beginning three minutes after the injection of said vaso-spastic pharmaceutical by which time the tissue mass oxygen levels will have been exhausted allowing the infused nitrogen gas to be transported into the mitochondria of said oxygen starved tissue mass, said infusion delivered through a pre-positioned, indwelling, microporous-tipped, catheter and continuing for a time period ranging from 10-15 minutes at a rate of from 0.1-0.2 ml/minute resulting in a total nitrogen gas dose ranging from 1.0 -3.0 millliliters, after which the catheter may be withdrawn and the procedure terminated.

2) The infusion of nitrogen gas as in claim 1, wherein the tissue mass is a solid malignant or non-malignant cancerous tumor.

3) The infusion of nitrogen gas as in claim 1, wherein the mass is an adipocyte containing fat deposit ranging in weight from 100-300 grams.

4) The infusion of nitrogen gas as in claim 3 wherein the procedure may be repeated in other areas of the body for as many treatments as deemed safe, necessary and useful.

5) The method of claim 2 wherein a tumor may be treated by means of the use of a small intraarterial balloon catheter for obtunding blood flow and infusion of nitrogen gas.
